SEQUENCE LISTING (9 sequences)

SEQ ID NO: 1 - DNA, 1101 bp, Human Mystique
atggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggaggacgcttgtgctatggagggaatgagattgtcactggaagctttggaggggatggtggagggcgccaagcggagggacaggaggaagaccaggagacccatccagccaagctggtga

SEQ ID NO: 2 - DNA, 1059 bp, Human Mystique
atggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctga

SEQ ID NO: 3 - DNA, 660 bp, Human Mystique
atggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcaggccgccctggaagccgacagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactga

SEQ ID NO: 4 - DNA, 186 bp, Human Mystique
atggcgttgacggtggatgtggccgggccagcgccctggggcttccgtatcacagggggcagggatttccacacgcccatcatggtgactaaggtctcaggctacgtctccagggcagaccaatggggacagctccttggaagtgctggcgactcgcttccagggctccgtgaggacatacactga

SEQ ID NO: 5 - DNA, 978 bp, Human Mystique
atggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaacaatggggacagctccttggaagtgctggcgactcgcttccagggctccgtgaggacatacactgagagtcagttctccttaaggtcctcctactccagcccaacctccctcagccgagggccggcagccccttctcaccaccaccctctagcagctccctcactggagaggcagccatcagccgcaggcggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctga

SEQ ID NO: 6 - DNA, 939 bp, Mouse Mystique
atggcgttgactgtggatgtggcaggaccagcaccttggggcttccgaattagcgggggcagagatttccacacacccatcattgtgaccaaggtcacagagcggggcaaggctgaagcagctgatctccggcctggcgacatcattgtggccatcaatggacagagtgcagagaacatgctacacgcggaggcccaaagcaagatccgacagagcgcctcacccctaagactgcagctggaccggtcccaaacagcctctcctgggcagaccaatggggagggctccttggaagtgctggcaaccagattccagggctccctgaggacacaccgtgacagccagtcttcccagagtttccagagtctgacacactctccaggccttgctgctgctcaccacttgacctaccctggccaccccaccagccaacaggccggccacagcagcccaagcgactccgcagtgagggtgctgctccattccccaggacggccctccagccctaggttcagcagtttggatctggaggaagactcagaggtgttcaagatgctgcaggagaaccgccagggacgggccgccccaaggcagtccagctcttttcgactcttacaggaagccttggaggctgaggagagaggtggcacacctgcctttgtgcccagctcgctgagctcccaggcttccttgcccacctccagggccttggccactccacccaagctccacacctgtgagaaatgcagcgtcaacatctcgaaccaggcggtccgcatccaggaggggaggtaccgacaccctggctgctacacttgcgcagactgtgggctgaacctgaagatgcgcggccacttctgggtgggcaatgagttgtactgcgagaagcatgcccgccagcgctactctatgcctggaactctcaactctcgagcctga

SEQ ID NO: 7 - RNA, 21 nt, Human Mystique
aagauccgccagagccccucg

SEQ ID NO: 8 - RNA, 21 nt, Human Mystique
aaucguggccaucaacgggga

SEQ ID NO: 9 - RNA, 21 nt, Mouse Mystique
aagauccgacagagcgccuca

(19) United States
(12) Patent Application Publication
(10) Pub. No.: US 2006/0177906 A1
(43) Pub. Date: Aug. 10, 2006

(54) IGF-I responsive gene and use thereof

(76) Inventors: Rosemary O'Connor, Carrigaline (IE); Gary Loughran, Carrigaline (IE)

Correspondence Address: JACOBSON HOLMAN PLLC, 400 SEVENTH STREET N.W., SUITE 600, WASHINGTON, DC, 20004, US

(21) Appl. No.: 11/405,121

(22) Filed: Apr. 13, 2006

Related U.S. Application Data
(63) Continuation of application No. PCT/IE04/00141, filed on Oct. 14, 2004.

(51) Int. Cl. C12P 21/06; C07H 21/04; C07K 14/72; C07K 14/65

(52) U.S. Cl. 435/69.1; 435/320.1; 435/325; 530/350; 530/399; 536/23.5

(57) ABSTRACT

A protein encoded by a gene comprises nucleic acid sequence SEQ ID No. 1, 2, 3, 4, 5, 6 or a derivative or mutant or fragment or variant or peptide thereof. The protein promotes the attachment and modulates the motility and invasion capability of cells.

### INTRODUCTION

The invention relates to a gene involved in the control of cell proliferation, survival, attachment, and movement.

Signals from receptor tyrosine kinases cooperate with adhesion signals to control cell proliferation, survival, and movement (1). Cancer cells acquire an enhanced ability to survive and migrate (2), but the mechanism of signalling integration between growth factor receptors and adhesion molecules is poorly understood.

IGF-I and IGF-II are ligands for the widely expressed IGF-I receptor tyrosine kinase, which promotes mitogenesis and cell survival (3). The IGF-I receptor (IGF-IR) is essential for normal growth during development, and also mediates powerful anti-apoptotic signals in response to diverse stimuli. Circulating IGFs and IGF-IR signalling pathways have also been associated with cancer progression (4). Increased expression of IGF-I, IGF-II, and the IGF-IR has been documented in many human malignancies and over-expression of the IGF-IR can confer cells with a transformed phenotype. Fibroblasts derived from IGF-IR knockout mice cannot be transformed by a series of oncogenes and transformation can be restored by re-expression of the IGF-IR. Inhibition of IGF-IR expression or signaling capacity by antibodies, triple helix formation, antisense strategies, or dominant negative mutants results in induction of apoptosis, failure to grow in anchorage-independent conditions, as well as inhibition of metastasis.

An emerging but important aspect of IGF-IR signalling coordinates inputs from integrins and other cell surface molecules to control cell motility and invasion in normal tissues and in tumour cell metastasis (5). In a mouse model of pancreatic islet cell tumourigenesis, endogenous IGF-IR expression was upregulated at invasive regions of the tumours, and ectopic IGF-IR expression resulted in tie accelerated development of highly invasive and metastatic carcinomas (6). Interestingly, the signals from the IGF-IR associated with survival and metastasis are associated with a domain in the C terminus of the receptor, (7-9), but the effectors of this domain are not yet known.

Cell motility and invasion are complex processes that require the coordination of signals from adhesion and growth-factor receptors. Signals from growth-factor receptors enhance or regulate adhesion and cell motility (5), by regulating actin organisation through the Rho kinases, by regulating the formation and disassembly of adhesion complexes with the extracellular matrix (ECM) through proteins such as focal adhesion kinase (FAK) (10), and by controlling the assembly of cell-cell contact through E cadherin-beta catenin complexes (11). However, the mechanisms of integration of IGF-IR signalling with integrin and ECM signalling are poorly understood. In particular, proteins that are activated by signals from growth factor receptors and that are necessary to mediate interactions with adhesion molecules or with proteins that are activated by signals from the extracellular matrix, are not known.

Identification and characterisation of these proteins would lead to an improved understanding of how cell motility is coordinated by signals from growth factor receptors and adhesion receptors and how cell motility and communication is controlled. These proteins would have valuable therapeutic potential in physiological and pathological conditions associated with cell movement and may be particularly important in conditions that are affected by growth factors or hormones, such as tumour cell metastasis, wound healing, tissue re-modelling, and inflammatory processes such as macrophage-mediated engulfment of microbes or killing of virally or bacterially infected cells.

### STATEMENTS OF INVENTION

According to the invention there is provided a protein encoded by a gene comprising nucleic acid sequence SEQ ID No. 1, 2, 3, 4, 5, 6 or a derivative or mutant or fragment or variant or peptide thereof.

In one embodiment of the invention the protein promotes the attachment and modulates the motility and invasion capability of cells.

In one embodiment of the invention the protein suppresses clonogenic growth of cells. The cells may be tumour cells.

In another embodiment of the invention the protein enhances β1 integrin activation and formation of fibrillar contacts with the ECM.

In one embodiment of the invention the protein has a PDZ-LIM domain.

The invention also provides an isolated DNA fragment comprising nucleic acid SEQ ID No. 3, SEQ ID No. 4 or SEQ ID No. 5.

The invention also provides isolated RNA oligonucleotides. (siRNA) comprising nucleic acid SEQ ID. No. 7 or 8. The invention also provides an isolated RNA oligonucleotide (siRNA) comprising nucleic acid SEQ ID. No 9 from mouse.

The invention provides Use of a nucleic acid sequence selected from any one or more of SEQ ID No. 1 to 6 or mutant or variant or SNP thereof as a diagnostic marker for cancer.

The invention further provides use of a protein or a derivative or mutant or fragment or variant or peptide thereof or DNA fragment or RNA oligonucleotide of the invention in controlling tumourigenesis, tumour cell motility and invasion, in wound healing and tissue repair or in organ remodelling or regeneration, vascular, immune and nervous system maturation or function.

The invention also provides use of a protein or a derivative or mutant or fragment or variant or peptide thereof or DNA fragment or RNA oligonucleotide of the invention as a predictive marker, in the diagnosis, treatment and/or prophylaxis of disorders characterised by inappropriate cell attachment, proliferation or survival or inappropriate cell death.

In one embodiment of the invention the disorder is selected from any one or more of inflammatory conditions, cancer including lymphomas and genotypic tumours. The disorder may also be selected from any one or more of autoimmune diseases, acquired immunodeficiency (AIDS), cell death due to radiation therapy or chemotherapy or acute hypoxic injury.

The invention also provides use of a protein or a derivative or mutant or fragment or variant or peptide thereof or DNA fragment or RNA oligonucleotide of the invention in the regulation and/or control of tumour cell metastasis or angiogenesis or in the modulation of the growth migratory, or attachment properties of cells in vivo and in tissue culture systems.

One aspect of the invention provides use of a protein encoded by a gene comprising nucleic acid sequence SEQ ID No. 2 or a derivative or mutant or fragment or variant or peptide thereof as a diagnostic marker for metastatic cancer.

Another aspect provides a medicament comprising a protein or DNA fragment or oligonucleotide of the invention.

The invention also provides a pharmaceutical composition comprising a protein or DNA fragment or RNA oligonucleotide of the invention and a pharmaceutically acceptable carrier thereof.

Methods for administration include those methods well known in the art such as oral, intravenous, intraperitoneal, intramuscular, transdermal, nasal, iontophoretic administration or the like The pharmaceutically acceptable carrier may be any commonly used carrier. In addition the dosage, dosage frequency and length of course of treatment may be determined or optimised by a person skilled in the art depending on the particular disorder being treated.

The invention further provides an immunogen comprising a protein or DNA fragment or RNA oligonucleotide of the invention.

The invention also provides monoclonal antibodies, polyclonal antibodies or antisera with specificity for a protein encoded by a gene comprising nucleic acid sequence SEQ ID No. 1, 2, 3, 4, 5, 6 or a derivative or mutant or fragment or variant or peptide thereof. Antibodies and antisera are generated using known procedures.

One aspect of the invention provides a diagnostic test kit comprising monoclonal antibodies, polyclonal antibodies or antisera or an immunogen of the invention.

The invention also provides a method of screening compounds for use in anti IGF-IR therapy comprising measuring the effect of the test compound on the expression levels of genes comprising nucleic acid SEQ ID No. 2 or nucleic acid SEQ ID No. 3.

The invention further provides a method of screening compounds for use as anti-cancer agents comprising measuring the effect of the test compound against Mystique activity in cells.

Suitable labels for use in screening assays according to the invention include a detectable label such as an enzyme, radioactive isotope, fluorescent compound or bioluminescent compound. Other suitable labels may be determined using routine experimentation. Furthermore the binding of the label may be accomplished using standard techniques known in the art.

The term derivative or mutant or fragment or variant or analogue or peptide, as used herein are understood to include any molecule or macromolecule consisting of a functional or characteristic portion of protein. Thus, functional equivalents of the protein may not share an identical amino acid sequence or composition.

### BRIEF DESCRIPTION OF THE DRAWINGS

The invention will be more clearly understood from the following description thereof, given by way of example only, in which:—

FIG. 1 shows the gene organisation of Mystique. The intron/exon organisation of three human Mystique variant cDNAs present in the databases. There are 12 exons numbered 1 to 12 and 11 introns. UTRs (untranslated region) are shown as open boxes, whereas CDS (coding sequences) are shown as black boxes and introns are depicted as black lines.

A schematic of each of the encoded protein isoforms is shown below with PDZ and LIM domains indicated.

FIG. 2A shows the alignment of the putative PDZ and LIM domains of human Mystique with those of its known homologues Reversion-induced LIM domain (RIL), Alpha-actinin-associated LIM protein (ALP) and CLP-36;

FIG. 2B shows a Northern blot analysis of R+ and R− cell RNA (left hand panel) and R+ cell RNA (right hand panel) that had been starved of serum before stimulation with IGF-I for the times indicated (0, 2, 4, 6 and 8 hours).

Blots were probed with the originally isolated partial cDNA for Mystique and then reprobed with 18s rDNA to measure loading;

FIG. 2C shows a mouse multiple tissue northern blot probed with Mystique cDNA and then with β-actin;

FIG. 2D shows a northern blot containing RNA from a series of human fibroblast and tumour cell lines probed with human Mystique cDNA;

FIG. 3A shows the immunofluoresence of HeLa cells transiently transfected separately with GFP-tagged Mystique isoforms;

FIG. 3B shows western blots generated from whole cell lysates (upper 2 panels) and detergent soluble and insoluble fractions (lower panel) derived from the indicated cell-lines and were probed with rabbit antiserum raised against the PDZ domain of human Mystique. Mystique 2 is the major immunoreactive protein at 39 kD;

FIG. 3C shows western blots prepared from whole cell lysates derived from R+ cells (top two panels) and DU-145 cells (bottom two panels) that had been starved of serum before stimulation with IGF-I for the indicated times. Blots were probed with the Mystique antiserum and an anti-β actin antibody;

FIG. 4A shows a western blot analysis of detergent insoluble fractions from Ha-Mystique stable transfectants of MCF-7 cells: M2A and B, Mystique 2; M3A and B, Mystique 3, Neo;

FIG. 4B is a graph showing the growth of MCF-7 stable transfectants in continuous monolayer culture without passaging;

FIG. 4C shows the immunofluoresence of stable clones of MCF-7 cell transfectants plated on collagen-coated plates and stained with anti-α-actinin antibody;

FIG. 4D is a graph showing invasion through matrigel and motility on collagen assayed in transwell plates for each cell-line;

FIG. 4E is a graph showing the results of a soft agar assay to monitor anchorage-independent cell growth;

FIG. 5 shows the immunofluoresence of MZA cells (MCF-7 cells stably expressing HA-tagged Mystique 2) grown on collagen-coated coverslips and then analysed by inmunofluoresence for expression of Mystique (with antibodies against HA or anti-Mystique antiserum as indicated) as well as expression of α-actinin, paxllin, β1 integrin or phosphotyrosine. as indicated. Inset panels showing co-localization of proteins indicate that Mystique does not colocalise with the focal adhesion markers paxillin and phosphotyrosine but does colocalise with a-actinin and activated β1 integrin;

FIG. 6A is a graph showing MCF-7 cells transfected with a human or mouse siRNA oligonucleotide and assayed for monolayer growth for six days after transfection. Untransfected cells were also assayed as a control. Inset: western blot analysis of lysates prepared from MCF-7 cells two and four days post transfection probed first with anti-Mystique antiserum and then with β-actin antibodies as loading control;

FIG. 6B is a graph showing cell viability determined by propidium iodide uptake over 6 days on MCF-7 cells transfected as in A;

FIG. 6C is a graph showing M2A and M3B cells assayed for their migration towards IGF-I in collagen-coated transwell plates two days after transfection of human or mouse siRNA oligonucleotides as indicated.

FIG. 6D shows the MCF10A cells assayed for their migration towards IGF-I in collagen-coated transwell plates two days after transfection of human or mouse siRNA oligonucleotides as indicated.

FIG. 6E shows the immunofluorescence analysis carried out on M2A cells two days after siRNA transfection. The upper panels shows immunolabelling of M2A cells transfected with the control mouse siRNA with anti-α-actinin and anti-HA antibodies. The middle panels shows immunolabelling of human siRNA transfected M2A cells with anti-α-actinin and HA antibodies. The bottom panels shows immunolabelling of siRNA transfected MZA cells with anti phosphotyrosine antibody and Mystique antiserum; and

FIG. 7 shows that over-expression of wildtype Mystique 2 (WT) enhances cell attachment of MCF-7 cells to the extracellular matrix material fibronectin. However, this enhancement of attachment is lost when the PDZ domain of Mystique is mutated at Leucine 80 to lysine (L80K). Enhancement of attachment is retained when the LIM domain critical structural cysteines at positions 313 and 316 are mutated to serine (CC313/316SS). A double mutant (L80K and CC313/316SS) does not promote cell attachment.

### DETAILED DESCRIPTION

We have identified an IGF-I responsive gene, Mystique, which encodes a novel PDZ-LIM domain protein that acts to integrate IGF-I Receptor (IGF-IR) and adhesion signalling. The gene Mystique has recently been renamed PDZLIM2. to indicate that it is one of a family of proteins that possess a PDZ and LIM domain.

The protein products of the Mystique gene have an essential function in regulating orgarisation of the cellular cytoskeleton, a function that is necessary for controlling cellular interactions with other cells and with the extracellular matrix (basement membrane, collagen, fibronectin etc). As a regulator of cytoskeletal organisation it may also control signals from adhesion molecules that are necessary for cell attachment, cell movement, cell growth, proliferation and cell survival.

Description of Mystique Variants

Table 1 lists Mystique gene variants and their encoded protein isoforms indicating the presence of predicted PDZ and/or LIM domains, amino acid length and Genbank accession number. A single variant of mouse Mystique 2 has been entered into publicly available databases although sequence alignment indicates that mouse Mystique is the ortholog of human Mystique 2.

TABLE 1

| | | | | protein | |
|---|---|---|---|---|---|
| Mystique variants | Species | PDZ | LIM | length (aa) | accession |
| Mystique 1 | Human | + | + | 366 | NP_789847 |
| Mystique 2 | Human | + | + | 352 | NP_067643 |
| Mystique 3 | Human | + | − | 219 | to be entered |
| Mystique 4 | Human | − | − | 64 | to be entered |
| Mystique 5 | Human | + | − | 278 | NP_932159 |
| Mouse Mystique | Mouse | + | + | 349 | NP_666090 |

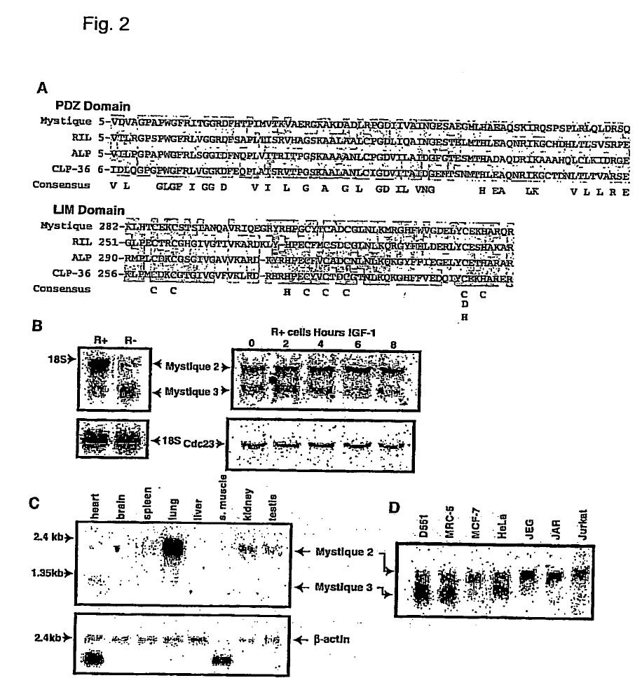

Homology searches revealed that Mystique shares homology with a small family of proteins that contain an N-terminal PDZ domain (12) and a C-terminal LIM domain (FIG. 2A). This family contains ALP Alpha actinin-associated LIM protein (13) from muscle; RIL (Reversion-induced LIM protein) (14) from fibroblasts; and CLP36 (15) from epithelial cells (16). These proteins share similarities with a family of LIM domain containing proteins including Enigma, Zyxin, and Cypher, which are proposed to function in regulating the actin cytoskeleton (17).

The PDZ domain is a protein interaction motif found in a diverse array of proteins (12, 18). It comprises approximately 85 amino acids and generally binds to the consensus sequence S/T-X-V/L/I normally found at the carboxyl terminus of target proteins. The PDZ domain binds to internal consensus sites, other PDZ domains, spectrin like repeats and LIM domains. The LIM domain is a double zinc finger structure found in homeodomain transcription factors, kinases, and other LIM proteins that can consist of several LIM domains (19). It can mediate protein-protein interactions and act to control gene expression in determining fate of cells during development LIM domains may also interact with kinases, phosphatases and cytoskeletal proteins to regulate their function (16, 20), and also have the potential to directly interact with DNA.

In order to identify genes associated with IGF-IR function in transformed cells, subtractive suppressive hybridisation (SSH) was used to isolate genes that were differentially expressed in a fibroblast cell line derived from the IGF-IR knockout mouse (R− cells) compared with R− cells that were re-transfected to express the IGF-IR (21) (R+ cells). From this screen we isolated a murine cDNA with homology to at least three different human cDNAs present in publicly available databases called Mystique. These cDNAs encode different proteins, which are splice variants of a single gene located on chromosome 8 (8p21.2).

Based on cDNA sequences (including ESTs) publicly available at NCBI [(National Center for Biotechnology Information, National Library of Medicine, Building, 38A, Bethesda, Md. 20894: (http://www.ncbi.nlm.nih.gov/entrez/query) three major human Mystique variants were identified.

Alignment of these three cDNAs with the human genome sequences indicates that all three variants are products of a single gene (˜20 kb) with 11 exons and 10 introns. As a result of alternative splicing, these Mystique variants are predicted to encode three different protein products. Mystique 1 and Mystique 2 are both predicted to code for proteins with one N-terminal PDZ domain and a single C-terminal LIM domain, with Mystique 1 only differing from Mystique 2 in the residues at the C-terminal side of the LIM domain. This difference at the C terminus may be important because Cuppen at al. (20) have shown that the region immediately C-terminal to the LIM domain of RIL (reversion-induced Lim gene), which is homologous to the Mystique LIM domain, is essential for its interaction with the PDZ domain of the phosphatase PTP-BL. The third major variant, which we designated Mystique 5 is predicted to encode a protein with an N-terminal PDZ, but the absence of exon 6 causes a frame shift in the remainder of the protein that results in premature termination and therefore, it encodes a protein of 25 kD.

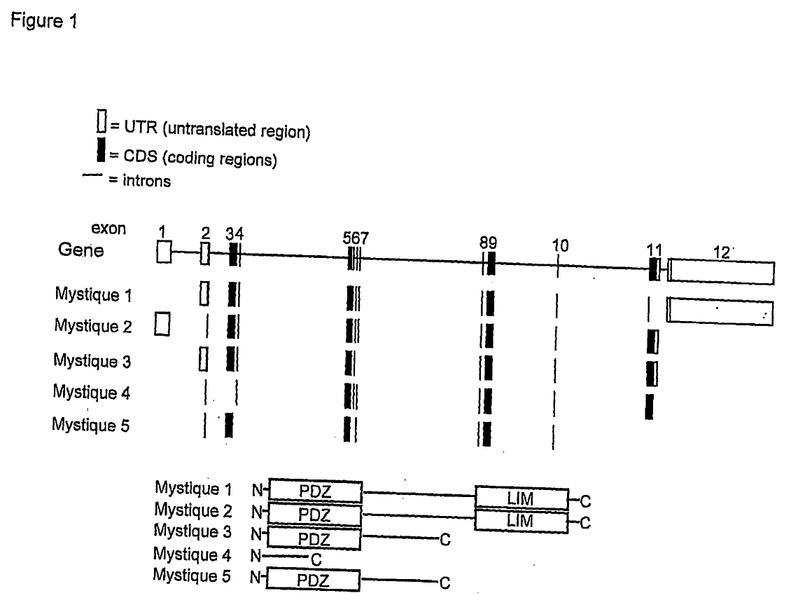

In addition we cloned by RT-PCR two additional human variants, which we designated Mystique 3 and 4. Mystique 3 is missing most of exon 6 and all of exon 7, which results in premature termination and it is predicted to encode a 27 kD protein isoform that includes the PDZ domain, but lacks the LIM domain (FIG. 1, FIG. 2B). Mystique 4 is missing exon 3, which results in premature termination and is predicted to encode a 10 kD protein isoform that lacks the PDZ domain and the LIM domain. FIG. 1 shows the gene organisation of the five Aystique cDNAs (Mystique 1, 2, 3, 4, and 5) and the intron-exon organisation of the Mystique gene. Northern blot analysis demonstrated that the Mystique clone isolated from the R+ cell-derived cDNA library hybridised with two distinct RNA species in R+ and R− cells (FIG. 2B). One transcript (˜1.8 kb) was more abundant in R+ cells and was confirmed by RT-PCR to be murine Mystique 2, whereas the other transcript (˜1.5 kb) was more abundant in R− cells and predicted to represent murine Mystique 3 or 5. Northern analysis of R+ cells (FIG. 2B) indicated that Mystique 2 mRNA accumulated in response to IGF-I stimulation whereas expression of Mystique 3 was repressed.

Alignment of human and mouse Mystique 2 shows that mouse Mystique is missing ˜350 bp of exon 1 which could account for the size discrepancy. In addition, RT-PCR on RNA extracted from R+ cells amplified a single band that corresponded in size and sequence to Mystique 2.

In a murine multiple tissue northern blot (FIG. 2C) Mystique 2 RNA expression was high in lung; moderate in kidney, testis, and spleen; low in heart, brain, and liver; and absent in skeletal muscle. In cell lines Mystique 2 RNA was present in MCF-7 breast carcinoma cells, HeLa cells, Jurkat T lymphocytic leukaemia cells and the JEG and JAR choriocarcinoma cells. Interestingly, Mystique 3 RNA was more abundant than Mystique 2 in heart and brain as well as in the MRC5 and D551 fibroblast cell lines and in HeLa cells (FIG. 2D). It appears from the RNA expression pattern that Mystique is expressed as alternatively spliced mRNA transcripts depending on cell type and on IGF-IR activation status.

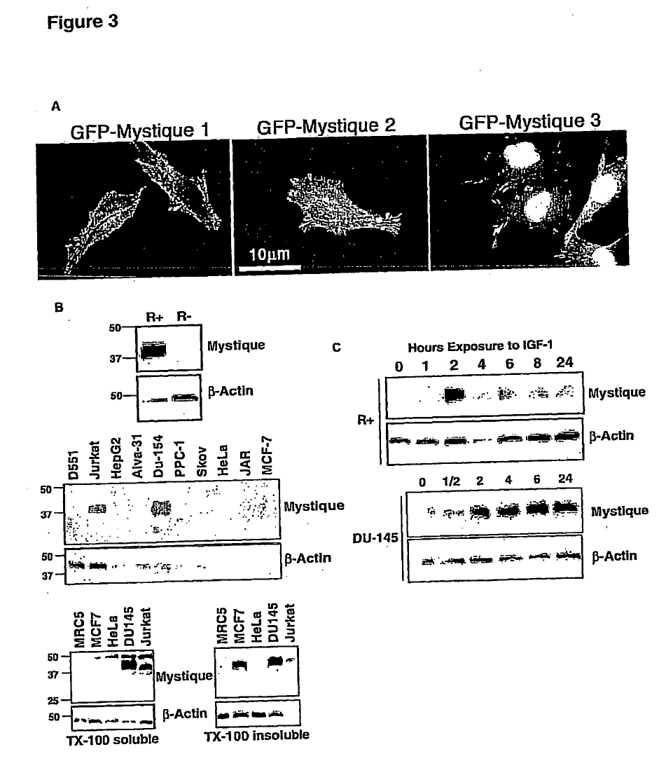

All three human Mystique cDNAs were cloned and used to generate expression constructs encoding GFP—(green fluorescent protein) or HA—(hemagglutinin) or His—(histidine) tagged fusion proteins. Transient expression of GFP-Mystique 1, 2, and 3 into HeLa cells demonstrated that Mystique 1 and 2 were predominantly localised at the cytoskeleton (FIG. 3A). By contrast, GFP-Mystique 3 was seen predominantly in the nucleus and only a minor amount was associated with the cytoskeleton. Similar patterns of staining were evident in cells transfected with HA-tagged Mystique constructs (not shown).

To detect endogenous Mystique protein a rabbit antiserum was generated against the PDZ domain, which is present in all isoforms. Western blot analysis of R+ and R− cells indicated a major immunoreactive band at 39k in R+ cells, which corresponds to the predicted size of Mystique 2 (FIG. 3B). This was not present in R− cells.

Mystique 2 was also detected in MCF-7 cells, Jurkat cells, Skov ovarian carcinoma, and DU145 prostate carcinoma cell lines, but was not detected in the D551 human fibroblast cell line or other cell lines shown in FIG. 3B; Mystique 2 was predominantly present in the detergent insoluble protein fraction in all cells, except for Jurkat cells, where it was exclusively present in the detergent soluble fraction and DU145 cells, where it was present in both fractions. Interestingly, a protein corresponding to the predicted size of Mystique 3 was not detected in any of the cell lines tested. This suggests that the Mystique 2 protein is expressed in cells where the Mystique 2 mRNA is more abundant than Mystique 3 mRNA (R+, MCF-7, JAR) but it is not expressed in cells where Mystique 3 mRNA is predominant (R−, D551, MRC5, HeLa) (FIG. 2D). Mystique 2 expression was also observed in leukocytes derived from normal blood and in leukemic cell lines.

Mystique expression was also examined in response to IGF-I stimulation and adhesion of R+, and DU145 cells (FIG. 3C). Mystique 2 levels were low in serum starved cells and were strongly induced in both cell lines after two hours IGF-I stimulation. Mystique 2 levels decreased by 6 hours IGF-I stimulation in R+ cells, and a similar pattern was seen in MCF-7 cells (not shown). However, Mystique 2 expression remained high in DU145 cells. This indicates that Mystique protein is transiently induced in response to IGF-I stimulation in R+ and MCF7 cells, but in the metastatic DU145 prostate cell line, which have much higher levels than any other cell lines tested, Mystique expression may be additionally stabilised or de-regulated. Mystique expression could not be induced by IGF-I in the fibroblast MRC5 cell line, which showed no basal expression. Interestingly, Mystique expression could be induced in MCF-7 and DU145 cells by adhering the cells to a substratum such as fibronectin and collagen. Furthermore Mystique expression could be induced upon differentiation of monocyte like cells into macrophages. Thus, Mystique expression is regulated by IGF-I, by adhesion signals, and by differentiation of cells, all of which indicates a particular role in cell spreading, attachment, and movement.

Since Mystique is an IGF-responsive gene (at both RNA and protein level) and it's expression is evident in fibroblasts that are transformed due to over expression of the IGF-IR, in epithelial cells, in tumour cell lines, and in leukocytes it could therefore, be a very useful biomarker for measuring IGF-1R activity or tumourigenic status. The chromosomal location of Mystique (8p21) is well documented as having genes that function in tumour suppression, in regulating tumourigenesis, and in metastasis. Currently there are many efforts underway in the Pharmaceutical industry to generate kinase inhibitors and anti-IGF-1R inhibitory antibodies as anti-cancer therapeutics. One of the challenges in drug development is to show in pre-clinical development, in clinical trials and with approved agent that the anti-IGF-1R therapeutic is effective. One way to do this is to have suitable biomarkers that indicate when IGF-1R activity is inhibited We have found that when the IGF-1R is not active that Mystique 2 expression levels (mRNA and protein) decline in tumour cells or any other cells that express it, and Mystique 3 (mRNA) increase. Thus, by measuring Mystique protein expression at the protein with antibodies, or by RT-PCR with suitable oligonucletide primers, or other detection methods, decreased Mystique 2 expression possibly increased Mystique 3 expression may be detected as an indicator of suppressed IGF-1R activity.

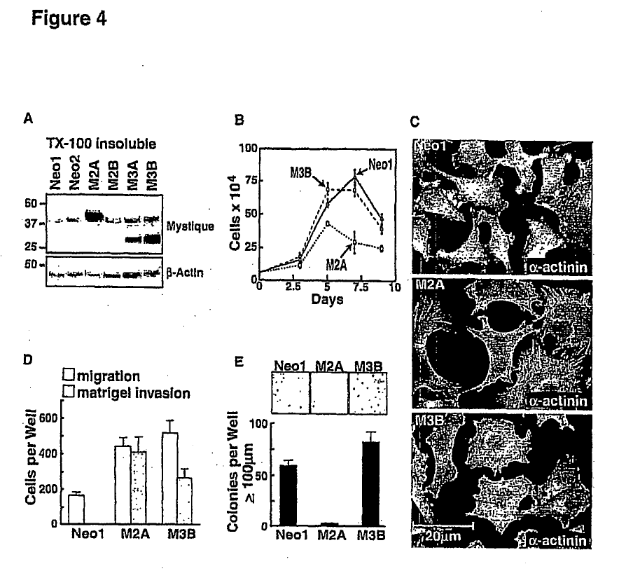

Since Mystique 2 localises to the cytoskeleton and is more abundant in transformed cells, we examined its ability to influence cell growth and motility. MCF-7 cells were stably transfected with either HA-Mystique 2 (M2) or HA-Mystique 3 (M3) (which served as a version of Mystique 2 lacking the LIM domain). Expression levels are shown for two clones each of Vector (Neo), Mystique 2 (M2), and Mystique 3 (M3) transfectants (FIG. 4A). Interestingly, endogenous levels of Mystique 2 were slightly increased in cells transfected with HA-Mystique 3. M2 cells and M3 cells had comparable short-term growth rates in monolayer culture (FIG. 3B). However, over longer times in culture (5-8 days) the saturation density of M2 cells was almost 50% lower than M3 or Neo cells. This indicates that M2 cells have retarded growth at higher confluence (FIG. 4B). The same growth pattern was evident with the other clones (not shown). M2 and M3 cells also displayed a more spread morphology compared with Neo cells. Immunolabelling of the cells with an anti-actinin antibody to visualize the cytoskeleton architecture indicated that M2 cells displayed a more organised cytoskeleton featuring prominent actin stress fibres and more abundant cell contacts with the ECM (FIG. 4C). This indicates that Mystique 2 in particular, but also Mystique 3, promotes organisation of the actin cytoskeleton and promotes cell contact with the ECM.

Cellular motility and the ability to invade into the ECM were examined by measuring cell migration in modified Boyden chambers and invasion into matrigel (FIG. 4D). Cells expressing high levels of either Mystique 2 or 3 (M2 and M3) cells exhibited increased migration compared with Neo cells. M2 and M3 cells both also acquired the ability to migrate into matrigel. Neo cells did not invade into matrigel. Cells over-expressing lower levels of Mystique demonstrated enhanced attachment to ECM, but little change in motility.

M2, M3, and Neo cells were also examined for their ability to form colonies in soft agarose. Results shown in FIG. 4E show that while M3 cells generated a higher number of colonies than Neo cells, M2 cells generated very small or undetectable colonies. Thus, overexpression of Mystique 2 suppresses clonogenic growth of MCF-7 cells while Mystique 3. slightly enhances clonogenic growth Clonogenic growth or the ability of cells to form colonies in soft agarose is a measure of anchorage-independent growth. This is growth without attachment to a plate or substratum that is a feature of transformed (cancer) cells that also form tumours in animals. These results indicate that the LIM domain, which is present in Mystique 2 and not in Mystique 3, functions to regulate cell growth at high confluence and can cause suppression of clonogenic growth, whereas the PDZ domain present in both Mystique 2 and 3 is associated with increased cell attachment, motility and invasion. Thus Mystique 2 or its LIM domain may have tumor suppression activity and Aystique may actually function as a tumour suppressor gene. Cells forced to over-express Mystique 2 may strongly interact with the the ECM and are thus less efficient at in anchorage-independent great and the formation of colonies in agarose.

Mystique 2 was found to be associated with Fibrillar adhesions, which are special contacts made between integrins and the extracellular matrix protein fibronectin (22, 23). These contacts are important for initiating the process of fibrillogenesis or laying down of fibrillar fibronectin around the cell. This in turn stimulates the organisation of other extraceuular matrix proteins including collagen and thus provides a matrix around the cell that facilitates movement, cell invasion and re-location (24). Fibrillogenesis is a necessary process in wound healing when fibroblasts move in to fill the wound and also for movement of cells during development of organs, in tissue regeneration and in angiogenesis. We have found that over-expression of Mystique promotes cell movement and invasion. Thus, over-expression of Mystique or derivatives of the protein or activating Mystique-interacting proteins may modulate or promote wound healing in vivo. Mystique may promote tissue repair, remodelling, and regeneration (by over expression locally) after surgery; or may promote repair of damage to organs, blood vessels, limbs or skin. A particular role in angiogenesis or in regeneration of neurons may also be predicted for Mystique because cell movement and invasion are necessary for the appropriate location and function of these tissues. The functions of Mystique also indicate potential roles in modulating tendon healing, fibrosis, cardiac remodelling and vascular remodelling in congenital cardiac disease.

The expression of Mystique in inflammatory leukocytes indicates a potential role in the cell movement, invasion and engulfment processes associated with macrophage or granulocyte engulfment of foreign bodies. It could also have a role in the movement, homing and target-directed killing function of T lymphocytes and natural killer cells.

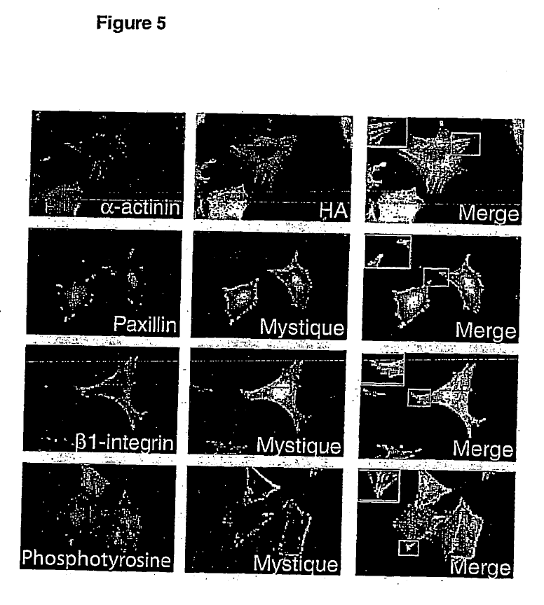

Mystique was found to co-localise with specific integrins or proteins associated with ECM adhesion complexes, which may explain the effects of Mystique on controlling cell growth and migration. MCF-7 cells over-expressing HA-Mystique 2 were immunolabelled with antibodies against either a actinin, paxillin, activated β1 integrin, or phosphotyrosine and counterstained with either the anti-HA antibody or the anti-Mystique antiserum. (FIG. 5). Mystique co-localised with α actinin at stress fibres but did not co-localize with paxillin or phosphotyrosine. Comprehensive colocalisation with β1 integrin was evident. Increased expression levels of activated β1 integrin were also evident in M2 cells, but not in M3 cells (not shown). This indicates that Mystique is not located at focal contacts with paxillin and phosphotyrosine but is instead located at fibrillar contacts, which contain activated α5β1 integrins but do not contain paxillin or phosphotyrosine (22). Fibrillar adhesions with the ECM promote remodelling of the ECM and facilitate cell movement (23, 24). Thus, the ability of Mystique 2 to promote the formation of fibrillar contacts together with its effects on organising the cytoskeleton may explain its profound effects on motility, invasion, and clonogenic growth.

These results indicate a clear difference in the function of the PDZ and LIM domains in cells and suggest that they interact with different cellular proteins to carry out these functions. The cellular location and function of Mystique 3 indicates that the PDZ domain (Mystique 3) appears to interact with cytoskeletal proteins including α actinin and is sufficient to promote motility and invasion, whereas the cellular location of Mystique 2 indicates that the LIM domain is present at the cytoskeleton and in fibrillar adhesions and may associate with integrins or other proteins that regulate the cytoskeleton and adhesion to carry out its functions.

Over-expression of Mystique may also be useful locally in cell or organ culture in vitro. For example it may be used in any cell or organ culture system where interaction with an extracellular matrix is necessary for the cells to grow and interact with one another properly. It would promote the propagation of skin layers developed for burn victims. It may also be useful in specialised culture conditions that are used to generate limb prostheses that are coated with material that interacts with bone and other tissues.

The association of Mystique with fibrillar adhesions and the high expression of Mystique mRNA in lung cells indicate another physiological function for Mystique in normal cells. This is the mechanical function of cell stretching, which is necessary for lung epithelial cells in order for lungs to fill with air, but is also necessary for cells to invade into tissues such as in metastasis and angiogenesis. Cell stretching requires contact with the extracellular matrix and is thought to be a dynamic process, but is difficult to study. If Mystique is involved in the stretching of lung cells it could be useful for the regeneration of lung epithelium that has lost it's ability to stretch. The presence of Mystique in lung also suggests that it may be associated with a hypoxic or oxygen response mechanism in cells. Hypoxia inducible genes are known to promote metastasis and invasion in cancer. Mystique RNA is also detectable in placenta and it is likely that it plays a role in the invasion or survival capacity of placental cells. Thus, Mystique or variants of Mystique or assays for Mystique function may be useful biomarkers or diagnostic agents for assessing placenta function and status in pregnancy. In general it is likely that Mystique has important functions during embryonic development that involve cell movement, invasion, and cellular responses to mechanical forces that can trigger gene expression necessary for the early cell layers to organize into tissues, to segregate, and generate organs.

Mystique may also have uses in commercial cell cultures. For example in a bioreactor environment that uses cell interacting with extracellular matrices that produce proteins of commercial interest over-expression of Mystique may modulate or enhance interaction with the matrices and thus modulate or enhance the culture life, viability and potential of the cells to produce protein. Mystique could also be used to propagate cells that may not normally grow in these bioreactor environments such as liver or lung epithelium.

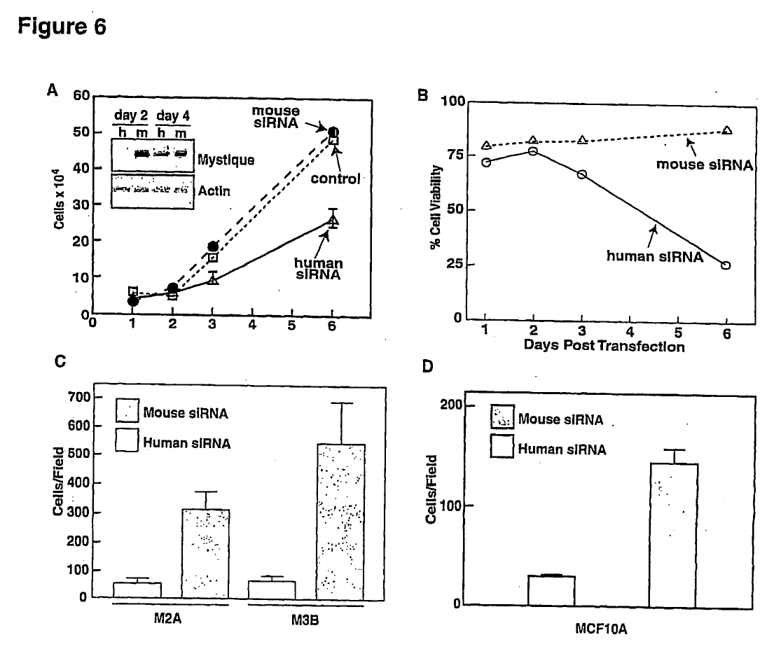

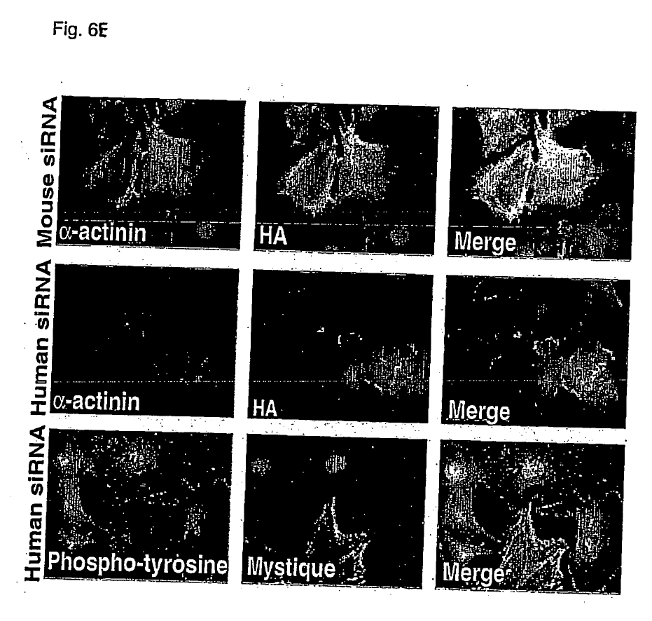

Since over-expression of both Mystique 2 and Mystique 3 promote cell attachment and modulate motility but only Mystique 2 regulates cell growth and inhibits growth in soft agarose, we examined if Mystique expression is necessary for maintaining cell architecture as well as for survival and growth. To address this issue, two different small inhibitory or interfering RNAs (siRNA) directed against Mystique were transfected into MCF-7 cells. SiRNA targeted against an equivalent sequence in the murine Mystique gene was used as a control. Two different human Mystique siRNAs abolished expression of endogenous Mystique 2 protein in MCF-7 cells by 48 hours. This was accompanied by reduced viability and retarded growth in monolayer culture when compared with cells transfected with control siRNA or untransfected cells. (FIGS. 6A and B). Interestingly, Mystique siRNA-treated cells had particular difficulty in attaching and surviving after being re-plated, which indicates an inability to interact with the ECM. Mystique siRNA also caused reduced expression of Mystique in the MCF-7 transfectants (M2 and M3 cells) and reversed the enhanced migratory capacity of these cells in Boyden chambers (FIG. 6C). Mystique siRNA oligonucleotides also suppressed endogenous Mystique expression in MCF-7 cells and other breast epithelial cell lines and almost completely suppressed migration of the cells into Boyden chambers (FIG. 6D). This indicates that Mystique is required for attachment and migration of epithelial cells. Mystique siRNA also caused a disruption of cytoskeletal actin organization as evidenced by the loss of the normal a-actinin staining pattern (FIG. 6E). This indicates that expression of Mystique is necessary to maintain cytoskeleton organisation, cell attachment to the ECM, as well as the survival, growth, and motility of cells. Importantly, transfection of Mystique siRNA into cells that do not express the protein (Rat 1 fibroblasts and human MRC5 fibroblasts) did not affect cell viability or growth. This confirms the selectivity of these siRNA oligonucleotides towards Mystique and suggests that an anti-Mystique therapeutic agent would selectivity kill or inhibit cells that express Mystique and would not damage cells that do not express Mystique.

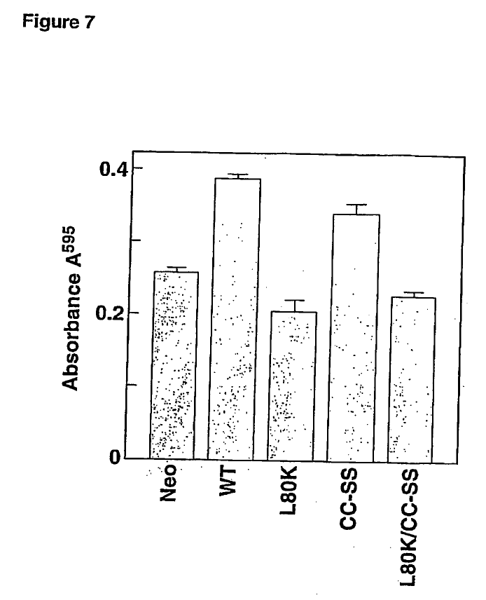

To assess the contribution of the PDZ and LIM domains to cell attachment and migration we generated mutants of Mystique 2 which had either the PDZ domain disrupted (L80K), the LIM domain disrupted (CC-SS), or both domains disrupted. These were compared with wild-type Mystique 2 (WT) in adhesion assays where cells were allowed to adhere to collagen or fibronectin (shown for fibronectin in FIG. 7). This demonstrated that while WT Mystique 2 promoted cell attachment the PDZ domain mutant did not promote cell attachment The LIM domain mutant promoted attachment slightly less well than WT, and the double mutant did not promote cell attachment. This indicates that a functional PDZ domain is essential to promote cell attachment, whereas the LIM domain is not essential for attachment These results are in agreement with our results for motility comparing Mystique 2 and Mystique 3 (Mystique3 lacks the LIM domain, but has the PDZ domain) where there is an equivalent enhancement of motility and invasion upon over-expression of these proteins.

Mystique mRNA is expressed as alternative spliced forms in IGF-IR knockout fibroblasts and IGF-IR-over-expressing cells. The Mystique protein is detectable in a series of transformed cell lines but not in IGF-IR knockout or fibroblast cell lines. Enforced expression of two isoforms of Mystique (Mystique 2 and 3) in MCF-7 cells promotes cell motility, invasion into matrigel, and suppresses clonogenic growth. The LIM domain may not be essential for modulating motility, but is essential for suppression of clonogenic growth because enforced expression of Mystique 2 causes suppressed clonogenic growth. This suggests that tumour suppressor activity is associated with the LIM domain. Mystique2 also enhances β1 integrin activation and formation of fibrillar contacts with the ECM. Conversely, knockdown of Mystique by RNA interference (siRNA) disrupts cytoskeletal architecture, suppresses the ability of cells to attach and grow, and also causes them to lose viability. Thus, Mystique expression is necessary for cell attachment, survival, and growth. Mystique functions to integrate signals from the IGF-IR with those mediated by β1 integrins to control growth and motility in transformed cells. Inhibition of Mystique expression appears to have a profound effect on tumour cell attachment, motility and survival and leads to induction of apoptosis. Thus Mystique siRNA is effectively an anti-tumour cell agent and is a potential anti-cancer therapeutic with potential to have particular activity in preventing metastasis. Mystique siRNA is also an anti-cell attachment and anti-cell migration agent that could be used in multiple other settings associated with cell movement, survival, proliferation and attachment, including angiogenesis, inflammation, hypertrophy, wound repair, and post surgical adhesions and tissue trauma.

Any other method of inhibiting or altering the balance of Mystique protein expression or function in cells would have a similar effect on cell survival, migration, attachment, proliferation. Such methods include but are not limited to the following; delivery of expression vectors that encode short hairpin inhibitory RNAs, antisense technologies, gene targeting, expression of dominant negative mutants of Mystique or particular isoforms or domains of Mystique, small molecule inhibitors, agents that bind to and disrupt the PDZ or LIM domains, or neutralising antibodies. In addition genes encoding Mystique binding partners (proteins, DNA, lipids etc) that are necessary for its function have the potential to have their function inhibited by siRNA and the other methods given above.

In order to express particular isoforms of the protein or domains of Mystique nucleotide sequences encoding different versions of the protein or fusion proteins may be generated and inserted into suitable expression vectors alone or as fusion proteins with GST (glutathione S-transferase, HA-(hemagglutinin), His-(histidine), fused peptides or as other fusion proteins

We have found that Mystique integrates signals from the IGF-IR with those mediated by β1 integrins that are known to regulate cell growth, motility, and invasion (25, 26). Signals from α5β1 integrins can cooperate with IGF-IR survival signalling (27) and promote interactions with endothelial cells, angiogenesis (28), and metastasis (29). It is noteworthy that Mystique 2 protein was more frequently detected in tumour cell lines and transformed fibroblasts, but not untransformed fibroblasts. Its enforced expression in the non-metastatic breast carcinoma MCF-7 cell line promoted a phenotype similar to that seen in highly metastatic tumour cell lines. This indicates that Mystique may promote epithelial/mesenchymal transitions, which entail the loss of polarised epithelial characteristics associated with development of mesenchyme. Similar changes are also observed late in the progression of human carcinomas (30). However, Mystique expression may not be limited to transformed cells and it may be also essential in normal blood cells and epithelial cells. Mystique expression in epithelial cells may be related to their tumourigenic, invasive and metastatic potential, and future studies are needed to test this hypothesis in primary tissues and in tumour models. Already the data obtained with cell lines suggest that Mystique 2 is a potential diagnostic marker for metastatic cancer and the anti-Mystique antiserum or other antibodies that measure Mystique expression would be useful diagnostic tools. Monoclonal antibodies that detect Mystique 2 or other isoforms can be generated by immunising mice with purified Mystique protein that was used to generate the rabbit antiserum and antibodies that define the different isoforms or domains of Mystique can be generated by immunising with purified proteins generated from expression constructs encoding the PDZ, LIM or other domains of the protein.

In addition to being used as diagnostic tools antibodies generated against Mystique may be used to track Mystique expression as a biomarker for tumours that are treated with agents that inhibit the activity of the IGF-1R or other growth factor receptors in cancer.

Genotyping and RT-PCR analysis of expression may also be used to assess Mystique expression as a predictive marker for cancer, metastasis, or angiogenesis. Other genetic analyses such as restriction fragment length polymorphism (RFLP) anlaysis or single nucleotide polymorphism (SNP) analysis may be used to detect Mystique or variants of Mystique as predictive markers for cancer, angiogenesis, inflammation, or other disorders associated with cell movement, attachment, survival, or IGF-1R function. SNPs could occur in the coding region, but may also occur in the non-coding region of the Mystique gene.

The promoter region of the Mystique gene may also be useful for assays directed at the use of Mystique as a predictive or diagnostic marker or for use as Mystique as a biomarker. Specific sites in the promoter region could be used either in the context of the Mystique sequence for analysis of transcriptional responses to IGF-I or other stimuli in cells, or as fusion proteins to generate reagents useful for measuring the transcriptional response to IGF-1R activation or possibly to activation of other growth factor receptors.

We have identified a possible mechanism of action of Mystique in signalling from examination of the functions of Mystique 2 and 3. Although the motility-promoting function of Mystique requires only the PDZ domain (as in Mystique 3) the effects on clonogenic growth and effects on β1 integrin function require the LIM domain. LIM domains from related proteins have been shown to bind to and regulate the activity of kinases including PKC isotypes, IR, and RET and also regulate transcription (20). Thus the LIM domain of Mystique may regulate the activity of functionally critical interacting proteins such as receptors, kinases, or other signalling proteins. Since Mystique 2 is rapidly and transiently induced in response to IGF-1 its LIM domain could mediate either signalling or transcription responses. Proteins that interact with Mystique (in particular kinases or phosphatases or other enzymes) may be critical mediators of signalling for cell survival, proliferation, motility and attachment. These proteins may be identified by using yeast two hybrid screens, recombinant or native Mystique proteins to fish out necessary binding partners from cells by immunoprecipitation or other protein “pull-down techniques”. Interacting proteins may be identified by sequence analysis of cDNAs that encode them, mass spectroscopy or other biophysical protein analysis methods including western blotting or peptide sequence analysis. A series of PDZ and LIM domain point mutants have been generated by PCR-based cloning to facilitate these studies. These mutations disrupt the unique structure and binding capacity of the PDZ and LIM domains and thus serve as controls for detection of proteins that specifically bind to the PDZ and LIM domains. Mystique-interacting proteins may themselves be important effectors of tumourigenesis, cell attachment, motility, inflammation, or IGF-1R signalling activity and have potential as useful diagnostic markers, biomarkers for drug activity, or targets for anti-cancer therapies. All of the protocols and methods for cloning, expression, and interacting protein studies may be found in text books such as those referenced (31, 32) and in the current literature that can be accessed through several databases including those referenced (33).

The present invention provides a means for limiting cancer-related deaths by controlling metastasis and angiogenesis. It provides a better understanding of the mechanism of cell attachment to the ECM or other cells, cell movement, stretching, and interactions with extracellular matrix in invasion. It provides a therapeutic potential for conditions associated with cell movement such as immune responses, cancer metastasis, wound healing, and tissue regeneration and re-modelling. It also provides therapeutic potential for modulating the survival or proliferation of any cells that are dependent on signals from adhesion and the cytoskeleton. Other disease states associated with cell survival, attachment, survival,and movement are intended to be included within the scope-of the invention.

The proteins and isolated DNA sequences of the invention may be used in the manufacture of medicaments for the treatment for such diseases. Any means well known to the skilled person in the field may be used to prepare such medicaments.

The Mystique protein may have the ability to translocate across membranes and act as a secreted protein that can enter nearby cells and interact with the cytoskeleton or nucleus. If Mystique has the intrinsic ability to cross membranes and localize to its site of action in cells this feature makes it useful as an agent that can be directly delivered to cells in order to mediate Mystique functions in cells. Alternatively, Mystique dominant negative or mutant forms could be delivered in this manner to inhibit endogenous Mystique activity in cells. A further use of a membrane translocation function in the Mystique protein would be to employ the necessary transport domains in Mystique or derivatives of them to carry other peptides or proteins or molecules into cells. Such a Mystique transporter function could be used in vitro or in vivo to deliver the protein itself or other agents into cells.

The invention will be more clearly understood by the following examples.

### EXAMPLES

Northern Blotting

Total RNA from 5×10⁶ cells was extracted using the Trizol Reagent (Gibco-BRL, Paisley, Scotland, UK) according to the manufacturer's instructions. Total RNA (20 μg) was separated by denaturing formaldehyde gel electrophoresis, transferred to nylon membranes, and immobilised by UV cross-linking (Stratalinker Stratagene, Amsterdam, Netherlands). Prehybridisation and hybridisation were carried out at 42° C. in 50% formamide, 5×SSC, 4× Denhardt's solution, 0.1% SDS, and salmon sperm DNA (100 μl/ml, Sigma Ireland, Dublin, Ireland) for 2 h and 15 h, respectively. ³²P-labelled probes (>1×10⁶ cpm/ml) were prepared by the random primer method (NEBlot: New England Biolabs, Hertfordshire, UK). Filters were washed twice at 42° C. in 2×SSC, 0.1% SDS for 5 ninutes, then twice at 42° C. in 0.1×SSC, 0.1% SDS for 15 minutes, and exposed to phosphorimager screens for empirically determined times. R+, R− and mouse multiple tissue northern blots (Clontech, BD Biosciences, Oxford, UK) were probed with the original mystique fragment isolated from the R+/R− subtracted cDNA library that corresponds to the 3′-UTR of mouse Mystique. The human multiple tumour northern blot was probed with a radiolabelled probe generated after XhoI digestion of the full coding sequence of human Mystique 2 from pcDNA3-HA-Mystique 2.

Cloning Mystique cDNAs.

Mystique was amplified by RT-PCR on total RNA extracted from MCF-7 cells using the following primers: MF 5′-cttctcgaggtatggcgttgacgg-3′; MR 5′-catctcgagctcaggcccgagag-3′. Two distinct products of ˜1.0 kb and ˜0.9 kb were amplified, purified and cloned using Xho1 (bold sequences in primers) into pcDNA3-HaX. Sequencing of inserts confirmed the larger insert (1.05 kb) to be Mystique 2 and the smaller insert to be two different splice variant of Mystique 2, which we called Mystique 3 and 4. As shown in FIG. 6 these splice variants are missing different exons and result in different protein products. Mouse Mystique 2 was amplified by RT-PCR on total RNA extracted from R+ cells using the primers MF and MR and cloned in the same way as the human fragments. Mutants of Mystique 2 (L80K and CC313/316SS) were generated by PCR using suitable oligonucleotides and were verified for harbouring the mutations by DNA sequencing.

Cell Culture and Transfection

R− cells are a mouse embryo fibroblast cell line derived from mice with a targeted disruption of the IGF-IR and R+ cells are R− cells that were transfected to express the IGF-IR (16). All cell lines were maintained in Dulbecco's modified Eagle's medium (DMEM: Biowhittaker UK, Berlcshire, UK) supplemented with 1 mM glutamine, 10% FBS and antibiotics.

For R+ versus R− cell RNA and protein extraction, cells were passaged 24 hours before harvesting for RNA or protein and were grown to approximately 70% confluence. For RNA and protein extraction from R+ cells stimulated with IGF-I (PeproTech, Rocky Hill, N.J.), cells were washed and starved from serum for 4 h before the addition of 100 ng/ml IGF-I (final concentration) for the indicated times.

For transient transfections of HeLa cells with GFP- or HA-tagged Mystique isoforms, cells were transfected with 4 μg of DNA using LipofectAMINE Plus, (Invitrogen). To generate stable transfectants of HA-Mystique 2 and HA-Mystique 3, MCF-7 cells were transfected with pcDNA3/HA-Mystique 2, pcDNA3/HA-Mystique 3. or empty pcDNA3 vectors. At 24 h after transfection cells were cultured in medium containing G418 (1 mg/ml) for 14 days, at which time individual clones were selected, expanded, and screened for expression of HA-Mystique by Western blotting. Clones of MCF-7 cells stably overexpressing HA-Mystique 2 or HA-Mystique 3 were maintained in DMEM supplemented with 1 mg/ml G418.

Mystique Antiserum

A restriction fragment encoding amino acids 1-184 (including PDZ domain) was cloned into pGEX-6P1 prokaryotic expression vector (Pharmacia). GST-fused 1-184 protein was purified by affinity chromatography and used to immunise a rabbit. Affinity-purified polyclonal antibodies were obtained by applying whole serum to nitrocellulose-immobilised GSt-fused 1-184 fragment. Bound antibodies were eluted with 500 μl 0.2 M glycine pH 2.15 and neutralised with 200 μl 1M K₂HPO₄ pH 7.0 before extensive dialysis against 1×PBS at 4° C.

Antibodies, Immunofluoresence and Western Blotting

Mouse anti-paxillin and anti-phosphotyrosine antibodies were purchased from Upstate Biotechnology. Mouse anti-actinin (BM75.2) and anti-β-actin antibodies were purchased from Sigma. Mouse anti-β1-integrin (12G10) was purchased from Serotec, Oxford, UK. Mouse Anti-HA (16B12) was purchased from BabCO, Berkeley, Calif.

For immunofluoresence, glass coverslips were coated with 10 μg/ml Collagen I (Sigma) at 4° C. overnight. Cells were then allowed to attach onto precoated coverslips for at least 12 h, rinsed with PHEM (60 mM Pipes, 25 mM Hepes, 10 mM EGTA, 2 mM MgCl₂; pH 6.9), fixed in 3.7% formaldehyde in PHEM for 10 minutes and permeablised with 0.1% Triton X-100 in PHEM for 5 minutes. After preblocking with 2.5% normal goat serum (NGS; Sigma) in PHEM for 30 minutes, cells were incubated with primary antibody, washed with PHEM and incubated with Cy2- or Cy3-conjugated secondary antibody (Jackson Labs.).

Whole cell lysates were prepared by lysing cells in ice-cold SDS-lysis buffer (1% Nonidet P40, 0.1% SDS, 20 mM Tris, 50 mM NaCl, 50 mM sodium fluoride, 1 μM pepstatin, 1 mM phenylmethylsulfonyl fluoride, 1 μM aprotinin, and 1 mM sodium orthovanadate, pH 7.6). Cell debris was removed by centrifugation at 15,000×g at 4° C. for 15 mins and samples were then denatured by boiling in 5×SDS-PAGE sample buffer for 5 minutes.

Detergent soluble fractions were prepared by lysing cells in ice-cold CSK extraction buffer (10 mM PIPES, pH 6.8, 100 mM NaCl, 300 mM sucrose, 3 mM MgCl₂, 1 mM EGTA) with 0.5% triton-X100 and protease inhibitors. Detergent insoluble material was pelleted by centrifugation and the pellets were resuspended in 2% SDS, 50 mM Tris pH 7.5.

Proteins were resolved using 4-20% gradient SDS-PAGE and transferred to nitrocellulose membranes (Schleicher & Schuell), which were blocked with 5% milk in TBS-T (20 mM Tris, 150 mM NaCl, 0.05% Tween 20, pH 7.6) for one hour at room temperature. Antibodies were diluted 1/1000 in TBS-T, 5% milk and incubated at 4° C. overnight. Horseradish peroxidase-conjugated secondary antibodies (Dako, Glostrup, Denmark) were used for detection using chemiluminescence with the ECL reagent (Amersham).

Proliferation, Survival and Soft Agar Assays

To measure proliferation in monolayer culture MCF-7 cell transfectants of Mystique 2, Mystique 3 or vector were cultured in Dulbecco's modified Eagle's medium supplemented with 10% fetal calf serum (complete medium) at 4×10⁴ cells per well in multiple wells of a 24-well plate. At intervals cells were removed from triplicate wells and counted using a haemocytometer. Data are presented as the mean and S.D. of counts from triplicate wells.

Cell viability was measured by resuspending cells in phosphate-buffered saline containing 1 μg/ml propidium iodide (Sigma). Samples were analysed by flow cytometry for the ability to exclude propidium iodide. The number of propidium iodide excluding cells was calculated and expressed as a percentage of the total number of cells.

Anchorage-independent growth was determined by assaying colony formation in soft agar. Cells were resuspended in 0.33% low-melting point agarose (Sigma) in DMEM/10% PBS onto a 35 nim dish containing a 2 ml base agarose layer (0.5%). The cells were fed every 3-4 days by adding 200 μl of DMEM/10% FBS. Colonies were counted and photographed after 5 weeks.

Migration and Matrigel Invasion Assays

MCF-7 cell transfectants (at or near confluency) were trypsinised and cultured in fresh media 12-16 h prior to each assay. Cells were harvested with non-enzymatic cell dispersant (Sigma), washed twice and then resuspended in DMEM containing 0.01% BSA (DMEM/BSA). The final cell density was determined using a haemocytometer. The lower wells of a collagen-coated Boyden chamber was loaded with DMEM/BSA long/mi IGF-I (final concentration). A 50 μl volume of cell suspension containing 50,000 cells was added to each upper well. The loaded chamber was placed in a 37° C. incubator enriched with 5% CO₂. After 4 h, the chamber was removed from the incubator and disassembled. Cells on the upper surface of the membrane were removed by scraping so that only cells that had migrated through the membrane remained. The membrane was then fixed with methanol, stained with 0.1% crystal violet and then air-dried. Cell counts were obtained by counting all cells and data are presented as average of counts from triplicate wells for each test condition.

siRNA Oligonucleotides and Transfection

Small interfering RNAs (siRNAs) targeted to human and mouse Mystique were obtained from Dharmacon with the following sequences: human Mystique; 5′-aagauccgccagagccccucg-3′; mouse Mystique; 5′- aagauccgacagagcgccuca-3′ (corresponding to nucleotides 199-219 after the start codon for both human and mouse Mystique). Nucleotides typed in bold indicate where the mouse siRNA differs from the human. A second human Mystique siRNA with the sequence aaucguggccaucaacgggga corresponding to nucleotides 144-164 after the start codon was also tested. MCF-7 cells (30-50% confluent) were transfected with 50 pmol (Z4 well plate) or 200 pmol (6 well plate) of oligonucleotide using the OligofectAMINE transfection reagent (Invitrogen). Cells were assayed for expression of protein by western blotting with the anti-Mystique antiserunm from 48-96 h after transfection, and assayed for growth, migration, survival and immunofluoresence analysis 48 h after transfection.

Adhesion Assay

Following a 4 hour serum starve, MCF-7 cells were removed form plates with trypsin/EDTA, counted and 2×10⁴ cells were plated onto 5 μg/ml fibronectin or collagen or laminin in quadruplicate wells of a 96-well plate and allowed to adhere for 30 minutes. Unattached cells were washed off plates with serum-free media and remaining cells were fixed and permeabilised with −20° C. methanol and then stained with 0.05% Crystal Violet. Stained cells were washed extensively before Crystal Violet extraction using 0.5% TX-100. Crystal violet was quantified by reading absorbance at A595 on a spectrophotometer

The invention is not limited to the embodiments herein before described which may be varied in detail.

### REFERENCES

1. Schwartz, M. A. & Ginsberg, M. H. Networks and crosstalk: integrin signalling spreads. Nat Cell Biol 4, E65-8 (2002).
2. Hanalian, D. & Weinberg, R. A. The hallmarks of cancer. Cell 100, 57-70 (2000).
3. Adams, T. E., Epa, V. C., Garrett, T. P. & Ward, C. W. Structure and function of the type 1 insulin-like growth factor receptor. Cell Mol Life Sci 57, 1050-93, (2000).
4. LeRoith, D. & Roberts, C. T., Jr. The insulin-like growth factor system and cancer. Cancer Lett 195,127-37 (2003).
5. Brooks, P. C. et al. Insulin-like growth factor receptor cooperates with integrin alpha v beta 5 to promote tumor cell dissemination in vivo. J Clin Invest 99, 1390-8. (1997).
6. Lopez, T. & Hanahan, D. Elevated levels of IGF-I receptor convey invasive and metastatic capability in a mouse model of pancreatic islet tumorigenesis. Cancer Cell 1, 339-53. (2002).
7. O'Connor, R. et al. Identification of domains of the insulin-like growth factor I receptor that are required for protection from apoptosis. Mol Cell Biol 17, 427-35. (1997).
8. Baserga, R. The IGF-I receptor in cancer research. Exp Cell Res 253, 1-6. (1999).
9. Brodt, P., Fallavollita, L., Khatib, A. M., Sarnani, A. A. & Zhang, D. Cooperative regulation of the invasive and metastatic phenotypes by different domains of the type I insulin-like growth factor receptor beta subunit J Biol Chem 276, 33608-15. (2001).
10. Casamassirna, A. & Rozengurt, E. Insulin-like growth factor I stimulates tyrosine phosphorylation of p130(Cas), focal adhesion kinase, and paxillin. Role of phosphatidylinositol 3′-kinase and formation of a p130(Cas).Crk complex. J Biol Chem 273, 26149-56. (1998).
11. Playford, M. P., Bicknell, D., Bodmer, W. P. & Macaulay, V. M. Insulin-like growth factor 1 regulates the location, stability, and transcriptional activity of beta-catenin. Proc Natl Acad Sci U S A 97, 12103-8. (2000).
12. Hung, A. Y. & Sheng, M. PDZ domains: structural modules for protein complex assembly. J Biol Chem 277, 5699-702. (2002).
13 Xia, H., Winokur, S. T., Kuo, W. L., Altherr, M. R. & Bredt, D. S. Actinin-associated LIM protein: identification of a domain interaction between PDZ and spectrin-like repeat motifs. J Cell Biol 139, 507-15. (1997).
14. Kiess, M. et al. Expression of ril, a novel LIM domain gene, is down-regulated in Hras-transformed cells and restored in phenotypic revertants. Oncogene 10, 61-8. (1995).
15. Wang, H., Harrison-Shostak, D. C., Lemasters, J. J. & Herman, B. Cloning of a rat cDNA encoding a novel LIM domain protein with high homology to rat RIL. Gene 165, 267-71. (1995).
16. Vallenius, T., Luukko, K. & Makela, T. P. CLP-36 PDZ-LIM protein associates with nonmuscle alpHA-actinin-1 and alpHA-actinin-4. J Biol Chem 275, 11100-5. (2000).
17. Khurana, T., Khurana, B. & Noegel, A. A. LIM proteins: association with the actin cytoskeleton. Protoplasma 219, 1-12. (2002).
18. Harris, B. Z., and Lirn, W. A. (2001) Mechanism and role of PDZ domains in signaling complex assembley. J. Cell Sci. 114: 3219-3231.
19. Bach, L (2000) The LIM domain: regulation by association. Mech of Dev. 91: 5-17.
20. Cuppen, E., Gerrits, H., Pepers, B., Wieringa, B., and Hendriks, W. (199S) PDZ motifs in PTP-BL and RIL bind to internal protein segments in the LIM domain protein RIL. Mol. Biol. Cell 9: 671-683.
21. Sell, C. et al. Effect of a null mutation of the insulin-like growth factor I receptor gene on growth and transformation of mouse embryo fibroblasts. Mol Cell Biol 14, 3604-12. (1994).
22. Zarnir, E. et al. Dynamics and segregation of cell-matrix adhesions in cultured fibroblasts. Nat Cell Biol 2, 191-6. (2000).
23. Dambere, T. et al. In vivo analyses of integrin beta I subunit function in fibronectin matrix assembly. J Cell Biol 110, 1813-23. (1990).
24. Sechler, J. L. & Schwarzbauer, J. E. Coordinated regulation of fibronectin fibril assembly and actin stress fiber fonnation. Cell Adhes Commun 4, 413-24. (1997).
25. Johnson, J. P. Cell adhesion molecules in the development and progression of malignant melanoma. Cancer Metastasis Rev 18, 345-57 (1999).
26. Morini, M. et al. The alpha 3 beta 1 integrin is associated with mammary carcinoma cell metastasis, invasion, and gelatinase B (MMP-9) activity. Int J Cancer 87, 336-42. (2000).
27. Zhang, H. et al. Beta 1-integnn protects hepatoma cells from chemotherapy induced apoptosis via a mitogen-activated protein kinase dependent pathway. Cancer 95, 896-906. (2002).
28. Klein, S. et al. Alpha 5 beta 1 integrin activates an NF-kappa B-dependent program of gene expression important for angiogenesis and inflammation. Mol Cell Biol 22, 5912-22. (2002).
29. Arboleda, M. J. et al. Overexpression of AKT2/protein kinase Beta leads to up-regulation of betal integrins, increased invasion, and metastasis of human breast and ovarian cancer cells. Cancer Res 63, 196-206. (2003).
30 Thiery, J. P. Epithelial-mesenchymal transitions in tumoutr progression. Nat Rev Cancer 2, 442-54. (2002).
31 Molecular Cloning: A Laboratory Manual. Third Edition. Editors: Sambrook and Russel. Cold Spring Harbor Laboratory, New York. (http://www.molecularcloning.com)
32. Protein-Protein Interactions (A Molecular Cloning Manual) Editor: E. Golemis (2002) Cold Spring Harbor Laboratory, New York.
33. Pubmed central (http://www.pubmedcentral.nih.gov/) or (http://www.ncbi.nlm.nih.gov/entrez/query.fcgi?db=pubmed&cmd=search&term=

1-28. (canceled)

29. A protein encoded by a gene comprising nucleic acid sequence SEQ ID No. 1, 2, 3, 4, 5, 6 or a derivative or mutant or fragment or variant or peptide thereof.

30. The protein as claimed in claim 29 which promotes the attachment and modulates the motility and invasion capability of cells.

31. The protein as claimed in claim 29 which suppresses clonogenic growth of cells.

32. The protein as claimed in claim 29 which enhances β1 integrin activation and formation of fibrillar contacts with the ECM.

33. The protein as claimed claim 29 having a PDZ-LIM domain.

34. An isolated DNA fragment comprising nucleic acid SEQ ID No. 3.

35. An isolated DNA fragment comprising nucleic acid SEQ ID No. 4.

36. An isolated DNA fragment comprising nucleic acid SEQ ID No. 5.

37. Isolated RNA oligonucleotides (siRNA) comprising nucleic acid SEQ ID. No. 7.

38. Isolated RNA oligonucleotides (siRNA) comprising nucleic acid SEQ ID. No. 8.

39. Isolated RNA oligonucleotide (siRNA) comprising nucleic acid SEQ ID. No. 9.

40. A method for detecting cancer comprising use of a nucleic acid sequence selected from any one or more of SEQ ID No. 1 to 6 or mutant or variant or SNP thereof as a diagnostic marker.

41. The method for controlling tumourigenesis, tumour cell motility and invasion comprising use of a protein or a derivative or mutant or fragment or variant or peptide thereof or DNA fragment or RNA oligonucleotide thereof as claimed in claim 29.

42. The method for wound healing and tissue repair comprising use of a protein or a derivative or mutant or fragment or variant or peptide thereof or DNA fragment or RNA oligonucleotide thereof as claimed in claim 29.

43. The method for organ remodelling or regeneration, vascular, immune and nervous system maturation or function comprising use of a protein or a derivative or mutant or fragment or variant or peptide thereof or DNA fragment or RNA oligonucleotide thereof as claimed in claim 29.

44. The method for the diagnosis, treatment and/or prophylaxis of disorders characterised by inappropriate cell attachment, proliferation or survival or inappropriate cell death comprising use of a protein or a derivative or mutant or fragment or variant or peptide thereof or DNA fragment or RNA oligonucleotide thereof as claimed in claim 29 as a predictive marker.

45. The method as claimed in claim 44 wherein the disorder is selected from any one or more of inflammatory conditions, cancer including lymphomas and genotypic tumours.

46. The method as claimed in claim 44 wherein the disorder is selected from any one or more of autoimmune diseases, acquired immunodeficiency (AIDS), cell death due to radiation therapy or chemotherapy or acute hypoxic injury.

47. The method for the regulation and/or control of tumour cell metastasis or angiogenesis comprising use of a protein or a derivative or mutant or fragment or variant or peptide thereof or DNA fragment or RNA oligonucleotide thereof as claimed in claim 29.

48. The method for the modulation of the growth or attachment properties of cells in tissue culture systems comprising use of a protein or a derivative or mutant or fragment or variant or peptide thereof or DNA fragment or RNA oligonucleotide thereof as claimed in claim 29.

49. A method for detecting metastatic cancer comprising use of a protein encoded by a gene comprising nucleic acid sequence SEQ ID No. 2 or a derivative or mutant or fragment or variant or peptide thereof as a diagnostic marker.

50. A medicament comprising a protein or DNA fragment or oligonucleotide thereof as claimed in claim 29.

51. A pharmaceutical composition comprising a protein or DNA fragment or RNA oligonucleotide thereof as claimed in claim 29 and a pharmaceutically acceptable carrier thereof.

52. An immunogen comprising a protein or DNA fragment or RNA oligonucleotide thereof as claimed in claim 29.

53. Monoclonal antibodies, polyclonal antibodies or antisera with specificity for a protein encoded by a gene comprising nucleic acid sequence SEQ ID No. 1, 2, 3, 4, 5, 6 or a derivative or mutant or fragment or variant or peptide thereof.

54. A diagnostic test kit comprising an immunogen as claimed in claim 52.

55. A diagnostic test kit comprising a monoclonal antibodies, polyclonal antibodies or antisera as claimed in claim 53.

56. A method of screening compounds for use in anti IGF-IR therapy comprising measuring the effect of the test compound on the expression levels of genes comprising nucleic acid SEQ ID No. 2 or nucleic acid SEQ ID No. 3.

57. A method of screening compounds for use as anti-cancer agents comprising measuring the effect of the test compound against Mystique activity in cells.
